SEQUENCE LISTING (1 sequence)

SEQ ID NO: 1 - DNA, 1475 bp, Pseudomonas aeruginosa NY3
cgctggcggcaggcctaacacatgcaagtcgagcggatgaagggagcttgctcctggattcagcggcggacgggtgagtaatgcctaggaatctgcctggtagtgggggataacgtccggaaacgggcgctaataccgcatacgtcctgagggagaaagtgggggatcttcggacctcacgctatcagatgagcctaggtcggattagctagttggtggggtaaaggcctaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccaacagaataagcaccggctaacttcgtgccagcagccgcggtaatacgaagggtgcaagcgttaatcggaattactgggcgtaaagcgcgcgtaggtggttcagcaagttggatgtgaaatccccgggctcaacctgggaactgcatccaaaactactgagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcgactagccgttgggatccttgagatcttagtggcgcagctaacgcgataagtcgaccgcctggggagtacggccgcaaggttaaaactcaaatgaattgacgggggcccgcacaagcggtggagcatgtggtttaattcgaagcaacgcgaagaaccttacctggccttgacatgctgagaactttccagagatggattggtgccttcgggagctcagacacaggtgctgcatggctgtcgtcagctcgtgtcgtgagatgttgggttaagtcccgtaacgagcgcaacccttgtccttagttaccagcacctcgggtgggcactctaaggagactgccggtgacaaaccggaggaaggtggggatgacgtcaagtcatcatggcccttacggccagggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccgcaagggggacggttaccacggagtgattcatgactggggtgaagtcgtaacaaggtagccgtag

(19) United States
(12) Patent Application Publication
(10) Pub. No.: US 2011/0306569 A1
(43) Pub. Date: Dec. 15, 2011

(54) RHAMNOLIPID BIOSURFACTANT FROM PSEUDOMONAS AERUGINOSA STRAIN NY3 AND METHODS OF USE

(75) Inventors: Xihou Yin, Corvallis, OR (US); Maiqian Nie, Xi'an (CN); Qirong Shen, Nanjing (CN)

(73) Assignees: Oregon State University; the State of Oregon Acting by and through the State Board of Higher Education on behalf of

(21) Appl. No.: 13/158,241

(22) Filed: Jun. 10, 2011

Related U.S. Application Data
(60) Provisional application No. 61/354,180, filed on Jun. 11, 2010.

(51) Int. Cl. A01N 43/04; C12P 7/64; C12N 1/20; C07H 15/06

(52) U.S. Cl. 514/25; 536/18.2; 435/134; 435/253.3

(57) ABSTRACT

The present disclosure relates to an isolated strain of Pseudomonas aeruginosa strain NY3 and compounds produced by this strain having biosurfactant activity, for instance rhamnolipids Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}, as well as compositions of, derived from, comprising, or consisting of one or more of such compounds isolated from P. aeruginosa. Also provided are methods of treating environmental materials contaminated with hydrocarbons, heavy metals, or pesticides with such compositions and methods of inhibiting microbial growth with such compositions.

### CROSS REFERENCE TO RELATED APPLICATION

This claims the benefit of U.S. Provisional Application No. 61/354,180, filed Jun. 11, 2010, which is incorporated by reference herein in its entirety.

### FIELD

The present disclosure relates to Pseudomonas aeruginosa having desirable biological activity and to rhamnolipids obtainable from such strains demonstrating the desirable biological activities, such as biosurfactant activities. The present disclosure further relates to compositions including rhamnolipid biosurfactants, as well as methods of making and using the compositions.

### BACKGROUND

Biosurfactants are surface-active amphipathic metabolites produced by a variety of microorganisms, including bacteria, fungi and yeasts. Glycolipids, phospholipids, lipopeptides/lipoproteins, fatty acids, and polymeric macromolecules are the main categories of structurally diverse biosurfactants (Desai and Banat, Microbiol. Mol. Biol. Rev. 61:47-64, 1997). They are primarily produced by fermentation with renewable carbon sources, such as vegetable oils (Costa et al., Process Biochem. 41:483-488, 2006). Their environmental compatibility, effectiveness at extremes of temperature, pH and salinity, and high specificity to targeted pathogens (Haba et al., J. Appl. Microbiol. 88:379-387, 2000; Rivardo et al., Appl. Microbiol. Biotechnol. 83:541-553, 2009) make biosurfactants attractive and desirable for widespread application. Applications for biosurfactants include for bioremediation (such as chelating heavy metals and/or improving bioavailability and degradation of pesticides, petroleum hydrocarbons, and polycyclic aromatic hydrocarbons), emulsifying and/or stabilizing agents (for example in food processing, cosmetic, or pharmaceuticals), wetting, foaming, and/or dispersing agents (for example in detergents and other cleaners), anti-adhesives (for example, preventing bacterial biofilm formation), and anti-microbial agents (such as anti-bacterial or anti-fungal agents).

Among the different classes of biosurfactants, rhamnolipids, members of the glycolipid group, are the most extensively studied and characterized (Desai and Banat, Microbiol. Mol. Biol. Rev. 61:47-61, 1997; Muthusamy et al., Curr. Sci. 94:736-747, 2008). Since rhamnolipids were first identified from Pseudomonas sp. (Jarvis and Johnson, J. Am. Chem. Soc. 71:4124-4126, 1949), chemical structures of some of these metabolites have been reported. An amphiphilic rhamnolipid molecule is composed of two moieties. One half is the hydrophilic sugar part, mono- or dirhamnose, and the hydrophobic lipid part possessing one or two fatty acid residues. These residues may either be both fully saturated or one may be saturated and the other unsaturated with either one or two double bonds. The lipid moiety is attached to the sugar by O-glycosidic linkage while the two 3-hydroxy acyl groups are joined together by the formation of an ester bond.

The structural diversity of rhamnolipids is determined by the number of rhamnose (one or two) and fatty acid (one or two), and the fatty acid components. The length of the constituent fatty acids has been found to vary from C₈ to C₁₄ and their combinations identified as: C₈₋₈, C₈-C₁₀, C₁₀-C₈, C₈-C_{10:1}, C₈-C_{12:1}, C_{12:1}-C₈, C₁₀-C₁₀, C₁₀-C_{10:1}, C₁₀-C₁₂, C₁₂-C₁₀, C₁₀-C_{12:1}, C_{12:1}-C₁₀, C₁₀-C_{14:1}, C_{14:1}-C₁₀, C₁₂-C₁₂, C₁₂-C_{12:1}, C_{12:1}-C₁₂, C₁₂-C₁₄, C₁₂-C_{14:1}, C_{14:1}-C₁₂, and C₁₄-C₁₄. Several single fatty acid-containing rhamnolipid compounds were also identified (Deziel et al., Biochim. Biophys. Acta 1440:244-252, 1999; Haba et al., J. Surfactants Detergents 6:155-161, 2003; Haba et al., Biotechnol. Bioeng. 81:316-322, 2003). In addition, novel mono and dirhamnolipid methyl esters (Rha-C₈-C₈ME and Rha-Rha-C₈-C₈ME) were described (Hirayama and Kato, FEBS Lett. 139:81-85, 1982). Rhamnolipids with alternative fatty acid chains have also been reported (Desai and Banat, Microbiol. Mol. Biol. Rev. 61:47-61, 1997). To date, over 40 different rhamnolipid components have been described, all having molecular masses below 800 Daltons. The Gram-negative opportunistic pathogenic bacteria Pseudomonas spp. were found to be the most common producers of rhamnolipids. Pseudomonas was also identified as one of the most frequently-isolated bacterial genera capable of degrading polycyclic aromatic hydrocarbons (PAHs), which are characterized as carcinogenic, mutagenic and ubiquitous environmental organic pollutants (Zhao and Wong, Environ. Technol. 30:291-299, 2009; Haritash and Kaushik, J. Hazard. Mater. 169:1-15, 2009).

### SUMMARY

The present disclosure relates to an isolated strain of Pseudomonas aeruginosa which is a soil bacterium. In an example, the isolated strain is Pseudomonas aeruginosa strain NY3.

The present disclosure also relates to compounds having biosurfactant activity, for instance rhamnolipids Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}, as well as compositions of, derived from, comprising, or consisting of one or more such compounds. In some examples, the one or more rhamnolipids are isolated from P. aeruginosa, such as P. aeruginosa strain NY3.

Also disclosed herein are methods of producing one or more rhamnolipids selected from Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}, such as a composition including one or more of such rhamnolipids. The methods include cultivating a rhamnolipid-producing microorganism (such as P. aeruginosa, for example P. aeruginosa strain NY3) under conditions wherein the one or more rhamnolipids are produced. In some examples, the one or more rhamnolipids are isolated from P. aeruginosa, such as isolated from the culture media.

Disclosed herein are methods of treating an environmental material (such as soil or water) contaminated with one or more hydrocarbon, heavy metal, or pesticide, including contacting the environmental material with an effective amount of a composition including one or more rhamnolipids selected from Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha C₁₀ₐ C_{10:1}, Rha-Rha-C₂₄, and Rha-Rha-C_{24:1}. In some examples, the hydrocarbon is a polycyclic aromatic hydrocarbon (PAH), such as fluorene, anthracene, phenanthrene, pyrene, or fluoranthene.

Also disclosed herein are methods of inhibiting microbial growth, including contacting the microbe with an effective amount of a composition including one or more rhamnolipids selected from Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}. In some examples, the microbe is a fungus (for example, Fusarium oxysporum) or a cyanobacterium (such as Synechocystis).

The foregoing and other features of the disclosure will become more apparent from the following detailed description, which proceeds with reference to the accompanying figures.

### BRIEF DESCRIPTION OF THE DRAWINGS

FIG. 1 is the nucleotide sequence of 16S rRNA isolated from P. aeruginosa strain NY3 (GenBank Accession No. GU377209).

FIG. 2 is a graph showing growth curves of P. aeruginosa strain NY3 on different carbon sources.

FIG. 3 is a graph showing time course of polycyclic aromatic hydrocarbon (PAH) degradation during fermentation of P. aeruginosa strain NY3.

FIGS. 4A to 4J are MALDI-TOF mass spectrometry spectra of rhamnolipid NY3BS samples isolated from fermentation using either glucose (FIGS. 4A to D) or glycerol (FIGS. 4E to J) as the sole carbon source.

FIGS. 5A and B are MALDI-TOF (FIG. 5A) and tandem mass spectrometry (FIG. 5B) spectra of a large molecular ion at m/z 1044.6.

FIGS. 6A and B are graphs of the effect of temperature (FIG. 6A) and NaCl concentration (FIG. 6B) on the surface tension of NY3BS.

FIG. 7 is a digital image showing growth of Fusarium oxysporum on potato dextrose agar plates in the presence of varying amounts of NY3BS preparation. Plate a, negative control; plates b and f, 1.4 mg NY3BS; plate c, 2.8 mg NY3BS; plate d, 4.2 mg NY3BS; plates e and g, 0 mg NY3BS.

### SEQUENCE LISTING

The nucleic acid sequences listed herein are shown using standard letter abbreviations for nucleotide bases. Only one strand of each nucleic acid sequence is shown, but the complementary strand is understood as included by any reference to the displayed strand.

The Sequence Listing is submitted as an ASCII text file in the form of the file named Sequence_Listing.txt, which was created on Jun. 8, 2011, and is 2,446 bytes, which is incorporated by reference herein.

SEQ ID NO: 1 is a nucleic acid sequence of 16S rRNA from Pseudomonas aeruginosa strain NY3.

### DETAILED DESCRIPTION

The present disclosure relates to an isolated strain of P. aeruginosa, designated strain NY3. This isolated bacterial strain produces biosurfactant substances, e.g., rhamnolipids, which have biological activities of commercial interest.

In an example, there is provided an isolate of P. aeruginosa strain NY3, which produces novel rhamnolipids. These rhamnolipids, or compositions including one or more of said rhamnolipids, can be used to decontaminate soil or water samples (for example to facilitate removal of PAHs, petroleum hydrocarbons, heavy metals, pesticides, or other environmental contaminants), and possess antimicrobial activities against organisms such as bacteria, fungi, and viruses. These substances can also be used as emulsifying, dispersing, foaming, wetting, and/or anti-adhesive agents in a variety of applications, including pharmaceutical formulations, detergents, cosmetics, and food processing.

### I. Abbreviations

BPLM/BSPM: biosurfactant production liquid medium

CMC: critical micelle concentration

PAH: polycyclic aromatic hydrocarbon

RBSSM: rhamnolipid biosurfactant-specific screening medium

Rha: rhamnose

### II. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. All sequence database accession numbers (such as GenBank, EMBL, or UniProt) mentioned herein are incorporated by reference in their entirety as present in the respective database on Jun. 10, 2011. In case of conflict, the present specification, including explanations of terms, will control. In addition, the materials, methods, and examples are illustrative only and are not intended to be limiting.

In order to facilitate review of the various embodiments of the invention, the following explanations of specific terms are provided:

Biosurfactant: A surface-active compound produced by a living cell (such as a microorganism, for example, a bacterium, fungus, or yeast). Properties of biosurfactants include reducing surface tension, forming or stabilizing emulsions, and promoting foaming. Biosurfactants are useful in bioremediation (for example, enhancing emulsification of hydrocarbons and increasing their bioavailability for microbial degradation), cosmetics and detergents, and as antimicrobial or antiviral agents. Biosurfactants are structurally diverse and include glycolipids, phospholipids, lipopeptides, fatty acids, and polymeric macromolecules. In one example, a biosurfactant is a rhamnolipid, such as a rhamnolipid produced by Pseudomonas aeruginosa (for example P. aeruginosa strain NY3 disclosed herein).

Cultivation: Intentional growth of a cell or organism, such as Pseudomonas aeruginosa, in the presence of assimilable sources of carbon, nitrogen and mineral salts. In an example, such growth can take place in a solid or semi-solid nutritive medium, or in a liquid medium in which the nutrients are dissolved or suspended. In a further example, the cultivation may take place on a surface or by submerged culture. The nutritive medium can be composed of complex nutrients or can be chemically defined.

Effective amount: An amount or dose sufficient to achieve a desired effect, such as treating a sample contaminated with hydrocarbons (for example, displacing or emulsifying the hydrocarbons), or having an anti-microbial effect (such as inhibiting growth or decreasing an amount of bacteria, cyanobacteria, or fungi in a sample, for example compared to a control). In some examples, an effective amount is a therapeutically effective amount, such as an amount or dose sufficient to achieve a desired effect in a subject or a cell being treated. For instance, this can be the amount of a composition including one or more rhamnolipids necessary to kill or inhibit growth of a microbe (such as bacteria, cyanobacteria, or fungus) in a subject or a sample.

Isolated: An “isolated” biological component (such as a rhamnolipid, nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Rhamnolipids that have been “isolated” include rhamnolipids purified by standard purification methods. For example, an isolated rhamnolipid can be a rhamnolipid that is substantially separated from other cell components, including other rhamnolipids. In some examples, an isolated rhamnolipid includes more than one rhamnolipid (for example a mixture of rhamnolipids), such as 2, 3, 4, 5, 6, 7, 8, 9, 10, or more rhamnolipids.

Pseudomonas aeruginosa: A Gram-negative, rod-shaped bacterium. It is found ubiquitously, including in soil, water, skin flora, plant surfaces, and surfaces in contact with soil or water. It is an aerobic organism, but is often considered to be a facultative anaerobe, as it can utilize nitrate as a terminal electron acceptor and can also ferment arginine. P. aeruginosa is an opportunistic pathogen of both humans and plants. It produces many compounds of potential commercial utility, including rhamnolipids, quinolones, phenazines, and lectins.

Purified: The term “purified” does not require absolute purity; rather, it is intended as a relative term. Thus, for example, a purified rhamnolipid preparation is one in which the rhamnolipid referred to is more pure than the rhamnolipid in its natural environment (such as within a cell or as secreted by a cell). For example, a preparation of a rhamnolipid (or a mixture of rhamnolipids) is purified such that the rhamnolipid (or the mixture of rhamnolipids) represents at least 50% of the total rhamnolipid content of the preparation.

Rhamnolipid: A glycolipid, generally including one or two rhamnose (Rha) molecules and one or two β-hydroxy fatty acids. A rhamnolipid with one rhamnose molecule is referred to as a mono-rhamnolipid, and a rhamnolipid with two rhamnose molecules is referred to as a di-rhamnolipid. The length of the fatty acids can include (but is not limited to) C₆ to C₂₄, such as C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄. The fatty acids may be saturated or unsaturated. A fatty acid is linked to a rhamnose by a glycoside linkage, and if present, a second fatty acid is linked to the first fatty acid by an ester bond.

Sample: A biological or non-biological material. In some examples, a biological sample includes material from an animal or plant source. Samples include biological samples such as those derived from a human or other animal source (for example, blood, stool, sera, urine, saliva, tears, tissue biopsy samples, surgical specimens, histology tissue samples, autopsy material, cellular smears, embryonic or fetal cells, amniocentesis or chorionic villus samples, etc.); bacterial or viral or other microbial preparations; cell cultures; forensic samples; agricultural products; plants or plant parts (such as leaves, stems, roots); waste or drinking water; milk or other processed foodstuff; and so forth. Non-biological samples include but are not limited to environmental materials, for example, water (such as groundwater, sea water, or water from a lake, river, stream, or other body of water), soil, or other items.

III. Isolated Pseudomonas aeruginosa

In the present disclosure, the isolation of a specific Pseudomonas aeruginosa strain that produces biosurfactant compounds of interest is disclosed. The strain was isolated from a soil sample contaminated with petroleum products and was selected based on production of rhamnolipids. Such selection methods involve culturing dilutions of contaminated soil in sterile water on nutrient media including N,N,N,-treimethyl-1-hexadecane ammonium bromide (CTAB) and methylene blue for a time sufficient to permit colony formation by a strain of P. aeruginosa associated with the soil sample and selecting one or more P. aeruginosa strains demonstrating production of rhamnolipids displaying a biosurfactant-indicating blue halo.

In an example, rhamnolipid-producing P. aeruginosa strain NY3 is isolated from a petroleum-contaminated soil sample. For example, P. aeruginosa strain NY3 produces rhamnolipids, including one or more rhamnolipids selected from Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}. In another example, P. aeruginosa strain NY3 produces the rhamnolipids listed in Table 3 (below), for example, when cultured by a 76 hour fermentation at 30° C. in medium containing (per liter): 5.0 ml phosphate buffer (25.82 g/L K₂HPO₃.3H₂O; 8.7 g/L KH₂PO₄; 33.4 g/L Na₂HPO₄.12H₂O; 5.0 g/L NH₄Cl), 3.0 ml MgSO₄ solution (22.5 g/L MgSO₄), 1.0 ml CaCl₂ solution (36.4 g/L CaCl₂), 1.0 ml FeCl₃ solution (0.25 g/L FeCl₃), 1.0 ml trace mineral elements (39.9 mg/L MnSO₄; 42.8 mg/L ZnSO₄.H₂O; 34.7 mg/L (NH₄)₆MO₇O₂₄.4H₂O), and 20 g/L glucose.

### IV. Rhamnolipid Biosurfactants

The present disclosure relates in certain embodiments to rhamnolipid biosurfactants. The rhamnolipid biosurfactants in various examples are the P. aeruginosa strain NY3, crude extracts obtained by cultivating the strain under culture conditions, or rhamnolipids isolated from the strain. In this manner the disclosure also provides novel rhamnolipid compounds and compositions including one or more novel rhamnolipids.

In some embodiments, the novel rhamnolipids have the following structures.

### Rha-C₈-C_{8:1}:

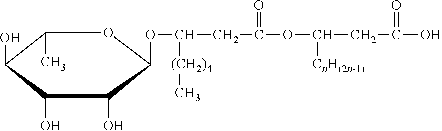

wherein n=5.

### Rha-C₁₆:

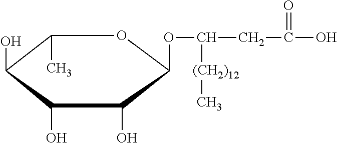

### Rha-C_{16:1}:

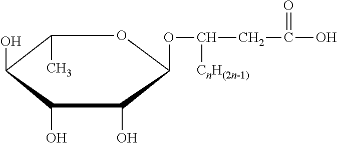

wherein n=13.

### Rha-C_{17:1}:

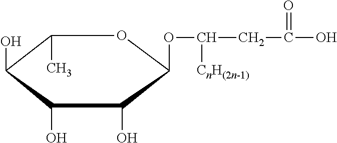

wherein n=14.

### Rha-C_{24:1}:

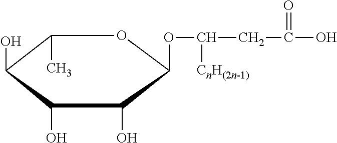

wherein n=21.

### Rha-Rha-C₆-C_{6:1}:

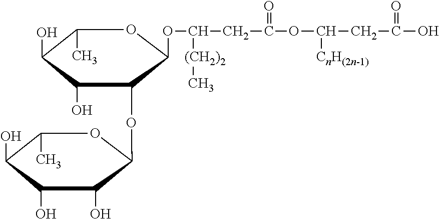

wherein n=3.

### Rha-Rha-C_{9:1}:

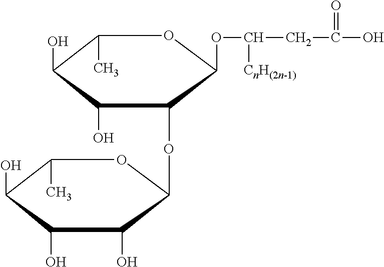

wherein n=6.

### Rha-Rha-C_{10:1}-C_{10:1}:

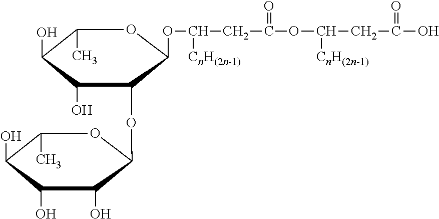

wherein n=7.

### Rha-Rha-C₂₄:

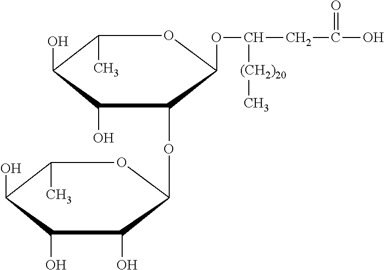

### Rha-Rha-C_{24:1}:

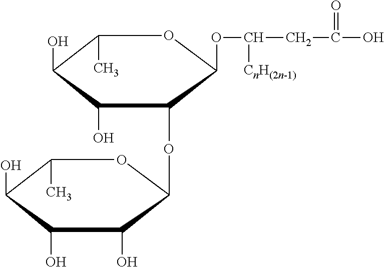

wherein n=21.

In other embodiments, the disclosed compositions include one or more rhamnolipids (such as 1, 2, 3, 4, 5, 6, 7, 8, 9, or 10 rhamnolipids) selected from Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}. In one example, the composition includes each of Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}. In other examples, the composition consists essentially of or consists of Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}. In some examples, the rhamnolipids are isolated from P. aeruginosa strain NY3. In further examples, the composition includes each of the rhamnolipids listed in Table 3 (below), for example, a rhamnolipid preparation isolated from P. aeruginosa strain NY3.

In some embodiments, the composition further includes additional compounds, such as one or more carriers, surfactants (such as a non-rhamnolipid surfactant), or biologically active agents (such as non-rhamnolipid biologically active agents, for example, a pharmaceutical agent or a non-rhamnolipid antimicrobial agent). One of skill in the art can select an appropriate carrier or other additional components based on the application of the rhamnolipid-containing composition.

In some examples, the composition includes a carrier, such as a pharmaceutically acceptable carrier. The pharmaceutically acceptable carriers useful in this disclosure are conventional. For example, Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the agents or compositions disclosed herein. In general, the nature of the pharmaceutically acceptable carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In other examples, the disclosed compositions may further comprise an inert material. Examples of inert materials include inorganic minerals such as diatomaceous earth, kaolin, mica, gypsum, fertilizer, phyllosilicates, carbonates, sulfates, or phosphates; organic materials such as sugars, starches, or cyclodextrins; or botanical materials such as wood products, cork, powdered corncobs, rice hulls, peanut hulls, or walnut shells.

In some embodiments, the compositions include a non-rhamnolipid surfactant. Examples of such surfactants include anionic surfactants such as carboxylates, for example, a metal carboxylate of a long chain fatty acid; N-acylsarcosinates; mono- or di-esters of phosphoric acid with fatty alcohol ethoxylates or salts of such esters; fatty alcohol sulfates such as sodium dodecyl sulfate, sodium octadecyl sulfate or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphthalene sulfonates, e.g., butyl naphthalene sulfonate; salts or sulfonated naphthalene-formaldehyde condensates; salts of sulfonated phenol-formaldehyde condensates; or more complex sulfonates such as amide sulfonates, e.g., the sulfonated condensation product of oleic acid and N-methyl taurine or the dialkyl sulfosuccinates, e.g., the sodium sulfonate or dioctyl succinate. Further examples of such surfactants are non-ionic surfactants such as condensation products of fatty acid esters, fatty alcohols, fatty acid amides or fatty-alkyl- or alkenyl-substituted phenols with ethylene oxide, block copolymers of ethylene oxide and propylene oxide, acetylenic glycols such as 2,4,7,9-tetraethyl-5-decyn-4,7-diol, or ethoxylated acetylenic glycols. Further examples of such surfactants are cationic surfactants such as aliphatic mono-, di-, or polyamine as an acetate, naphthenates or oleates; oxygen-containing amines such as an amine oxide of polyoxyethylene alkylamine; amide-linked amines prepared by the condensation of a carboxylic acid with a di- or polyamine; or quaternary ammonium salts.

In further embodiments, the compositions may include a deposition agent, which assists in preventing the composition from drifting or blowing away from a surface following deposition. Examples of useful deposition agents include, but are not limited to, soy protein, potato protein, soy flour, potato flour, fish meal, bone meal, yeast extract, and blood meal. Alternative deposition agents include modified cellulose (carboxymethylcellulose), botanicals (grain flours, ground plant parts), non-phyllosilites (talc, vermiculite, diatomaceous earth), natural clays (attapulgite, bentonite, kaolinite, montmorillonite), and synthetic clays (Laponite). The compositions may further include an antifreeze/humectant agent which suppresses the freeze point of the product and helps minimize evaporation when sprayed. Examples of antifreeze/humectant agents include, but are not limited to, ethylene glycol, propylene glycol, dipropylene glycol, glycerol, butylene glycols, pentylene glycols and hexylene glycols.

In other embodiments, the composition includes one or more cosmetics, pharmaceutical agents, food or food additives, or antimicrobial agents (such as an antibiotic or antimycotic agent). See, e.g., U.S. Pat. Publication Nos. 2010/0249058; 2007/0207930; 2007/0191292; 2006/0233935; U.S. Pat. No. 7,939,489.

### V. Methods of Producing Rhamnolipid Biosurfactants

Disclosed herein are methods of producing one or more of the disclosed rhamnolipids. In some embodiments, the methods include cultivating P. aeruginosa under conditions sufficient to produce one or more rhamnolipids selected from Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}. In some examples, the methods include cultivating P. aeruginosa strain NY3, disclosed herein. In one example, the methods include cultivating P. aeruginosa under conditions sufficient to produce the rhamnolipids listed in Table 3 (below).

Representative methods include cultivating a strain of Pseudomonas aeruginosa (e.g., P. aeruginosa strain NY3) and recovering the cells or one or more rhamnolipids from the culture medium. It may be desirable thereafter to form the free acid or a salt or ester by methods known by one of ordinary skill in the art.

In an example, P. aeruginosa strain NY3 is cultivated in a nutrient medium suitable for production of rhamnolipids using methods known in the art. For example, the cell may be cultivated by shake flask cultivation, small-scale or large-scale fermentation (including continuous, batch, fed-batch, or solid state fermentations) in laboratory or industrial fermenters performed in a suitable medium and under conditions allowing one or more rhamnolipids to be expressed and/or isolated. The cultivation takes place in a suitable nutrient medium comprising carbon and nitrogen sources and inorganic salts, using procedures known in the art. Suitable media are available from commercial suppliers or can be prepared according to published compositions (e.g., in catalogues of the American Type Culture Collection).

In one example, the nutrient media for the cultivation of the P. aeruginosa contains, in the range of about 0.1 to about 10%, a complex organic nitrogen source such as yeast extract, corn steep liquor, vegetable protein, seed protein, hydrolysates of such proteins, milk protein hydrolysates, fish and meat extracts, and hydrolysates such as peptones. In an alternative example, chemically defined sources of nitrogen can be used such as urea, amides, single or mixtures of common amino acids such as valine, asparagine, glutamic acid, proline, and phenylalanine. In further examples, carbohydrates (0.1-5%) are included in the nutrient media and starch or starch hydrolysates such as dextrin, sucrose, lactose or other sugars or glycerol or glycerol esters may also be used. The source of carbon can be derived from vegetable oils or animal fats (such as beef extract). In some examples, the medium includes a single carbon source, for example glucose, glycerol, beef extract, hexane, octane, or diesel oil.

In an example, mineral salts such as NaCl, KCl, MgCl₂, ZnCl₂, FeCl₃, CaCl₂, Na₂SO₄, FeSO₄, MgSO₄ and Na⁺ or K⁺ salts of phosphoric acid are added to the media described above particularly if chemically defined. In further examples, CaCO₃ (as a source of Ca⁺⁺ ions or for its buffering action), salts of trace elements (such as nickel, cobalt, zinc, molybdenum, or manganese) or vitamins are added to the media. The pH of the media is adjusted to be suitable for cultivation of P. aeruginosa. In some examples, the initial pH of the media is from about 2.0 to 10.0 (such as about 2.0, 2.5, 3.0, 3.5, 4.0, 4.5, 5.0, 5.5, 6.0, 6.5, 7.0, 7.5, 8.0. 8.5, 9.0, 9.5, or 10.0). In one example, the initial pH of the media is about 9.0.

In a particular non-limiting example, P. aeruginosa strain NY3 is cultivated in liquid media as shown in Table 1. The culture is incubated at about 30° C. with shaking (for example at about 100-300 rpm, such as about 200 rpm) for 12 to 102 hours. In one example, the P. aeruginosa strain NY3 is cultivated for about 76 hours prior to isolating the rhamnolipids. In one example, the initial pH of the media is about 9.0.

TABLE 1
Exemplary P. aeruginosa biosurfactant production liquid medium

| Component | Stock Solution Concentration | Amount/Liter Media | |
|---|---|---|---|
| Phosphate | 25.82 g/L K₂HPO₄•3 H₂O | 5.0 | ml |
| buffer | 8.7 g/L KH₂PO₄ | | |
| | 33.4 g/L Na₂HPO₄•12 H₂O | | |
| | 5.0 g/L NH₄Cl | | |
| MgSO₄ | 22.5 g/L | 3.0 | ml |
| CaCl₂ | 36.4 g/L | 1.0 | ml |
| FeCl₃ | 0.25 g/L | 1.0 | ml |
| Trace mineral | 39.9 mg/L MnSO₄ | 1.0 | ml |
| elements | 42.8 mg/L ZnSO₄•H₂O | | |
| | 34.7 mg/L (NH₄)₆Mo₇O₂₄•4 H₂O | | |
| Glucose | | 20 | g/L |

The present disclosure also relates to methods for obtaining an “isolated” preparation of one or more rhamnolipids. In an example, rhamnolipids are extracted from the culture supernatant or filtrate by a variety of methods known to the art. In a specific example, the cells of the P. aeruginosa are first removed from the fermentation by filtration or centrifugation before such extraction procedures are commenced. Precipitation may be by solvent extraction from culture filtrate, which may use an adjustment to acid pH values (such as acidification to about pH 2.0 with HCl). The precipitate is recovered, for example by centrifugation and extracted with an organic solvent, such as CH₂Cl₂, ethanol, methanol, or a combination thereof. Other primary methods of isolation which may be used include conventional methods such as adsorption onto carbon, precipitation, salting out, molecular filtration, or any method known in the art. In some examples, the yield of rhamnolipids utilizing the methods disclosed herein is from about 1 mg/L to about 50 g/L, for example, about 10 mg/L to about 25 g/L, about 20 mg/L to about 20 g/L, or about 50 mg/L to about 10 g/L.

### VI. Uses of Rhamnolipid Biosurfactants

Provided herein are compositions and methods of treating an environmental material (such as soil or water) contaminated with hydrocarbons (such as PAH or petroleum hydrocarbons), heavy metals (for example, cadmium, lead, or zinc), and/or pesticides (such as atrazine, trifluralin, coumaphos, or diuron). The methods include contacting the environmental material with an effective amount of a composition including one or more rhamnolipids disclosed herein (such as one or more of Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}). The environmental material can include soil (such as soil or sediment), water (such as ground water, surface water, sea water, or industrial or agricultural waste water), or sludge (such as industrial or agricultural sludge). In some examples, the environmental material is contacted with about 0.01% to about 5% (w/w or w/v) of a composition including one or more of the disclosed rhamnolipids, for example, about 0.01% to about 2.5%, or about 0.1% to about 0.5%. In some examples, the amount of a composition including one or more of the disclosed rhamnolipids is about 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.1%, 1.2%, 1.3%, 1.4%, 1.5%, 2%, 3%, 4%, 5%, or more.

In a particular example, soil which is contaminated with PAHs (such as fluorene, anthracene, fluoranthene, phenanthrene, pyrene, acenaphthylene, acenaphthene, benzanthracene, benzopyrene, benzofluoranthene, chrysene, coronene, and/or dibenzanthracene) is contacted with a composition including one or more of the disclosed rhamnolipids (such as one or more of Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}) in an amount sufficient to treat the contamination (such as about 0.1% to about 0.5% rhamnolipid). In other examples, soil which is contaminated with petroleum hydrocarbons is similarly treated with a composition including one or more of the disclosed rhamnolipids in an amount sufficient to treat the contamination. In some examples, the method includes contacting the soil with a rhamnolipid preparation isolated from P. aeruginosa strain NY3, prepared as described in Example 1, for example, a crude preparation of NY3 biosurfactants, or an isolated preparation of NY3 biosurfactants (for example, a composition including the rhamnolipids shown in Table 3). Without being bound by theory, it is believed that contacting an environmental material (such as soil or water) with one or more of the disclosed rhamnolipids emulsify and/or disperse the hydrocarbons and facilitate metabolism of the hydrocarbons by microbes in the environment (including, but not limited to P. aeruginosa).

In another particular example, water which is contaminated with PAHs (such as fluorene, anthracene, fluoranthene, phenanthrene, pyrene, acenaphthylene, acenaphthene, benzanthracene, benzopyrene, benzofluoranthene, chrysene, coronene, and/or dibenzanthracene) is contacted with a composition including one or more of the disclosed rhamnolipids (such as one or more of Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}) in an amount sufficient to treat the contamination (such as about 0.1% to about 0.5% rhamnolipid). In other examples, water which is contaminated with petroleum hydrocarbons is similarly treated with a composition including one or more of the disclosed rhamnolipids in an amount sufficient to treat the contamination. In some examples, the method includes contacting the soil with a rhamnolipid preparation isolated from P. aeruginosa strain NY3, prepared as described in Example 1, for example, a crude preparation of NY3 biosurfactants, or an isolated preparation of Ny3 biosurfactants (for example, a composition including the rhamnolipids shown in Table 3).

Also provided are compositions and methods of inhibiting microbial growth (such as bacterial or fungal growth), which include contacting the microbe (such as a sample including the microbe or a subject infected with the microbe) with an effective amount of a composition including one or more rhamnolipids disclosed herein (such as one or more of Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}). In some examples, the methods include treating or inhibiting a microbial infection in an organism, such as a plant or mammal, which include administering to the organism a therapeutically effective amount of a composition including one or more rhamnolipids disclosed herein (such as one or more of Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}), or a salt or ester thereof. The compositions can also be used to protect against viral pathogens, or against an array of invertebrate pathogens. In some examples, the methods include inhibiting fungal growth or treating a fungal infection, such as Fusarium, Aspergillus, Penicillium, Mucor, Gliocadium, or Chaetonium. In a particular example, the fungus is Fusarium oxysporum. In other examples, the methods include inhibiting bacterial growth or treating a bacterial infection, such as Serratia, Enterobacter, Klebsiella, Staphylococcus, or Bacillus. In general, an effective amount is a dose between about 0.1 and about 100 mg/kg. A preferred dose is from about 1 to about 60 mg/kg of active compound. In some examples, a typical dose is from about 7.5 mg to about 125 mg. One of skill in the art can select an appropriate dose based on the organism, type and severity of infection, and so on.

In additional examples, disclosed herein are methods of inhibiting growth of algae or cyanobacteria in an environmental material, such as water (for example, a lake, pond, tank, and so on). The methods include contacting the water with an effective amount of a composition including one or more rhamnolipids disclosed herein (such as one or more of Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}). In some examples, the methods include treating or inhibiting growth of cyanobacteria (such as Synechocystis sp., Synechococcus sp., Spirulina sp., Anabaena sp., Trichodesmium, Crocosphaera, and Arthrospira maxima). In general, an effective amount is about 0.01 mg/ml to about 100 mg/ml (such as about 0.1 mg/ml to about 10 mg/ml, or about 1 mg/ml).

The following non-limiting examples are provided to illustrate certain particular features and/or embodiments.

### EXAMPLES

### Example 1

### Materials and Methods

Chemicals: Pyrene (99%) was purchased from Sigma-Aldrich (Shanghai, China), phenanthrene from the Chemical Store of the Chinese Academy of Military Medical Sciences (Beijing, China), anthracene from Beijing Chemical Industry Co., fluorene (98%) from Johnson Matthey Co. (Shanghai, China) and fluoranthene from Tokyo Chemical Industry Co. (Shanghai, China). Unless otherwise stated, the organic solvents, media and medium ingredients, salts, and acids were purchased from Sigma-Aldrich, VWR, or Fisher, USA.

Screening and isolation of the biosurfactant-producing bacterial strains: Petroleum-contaminated soil samples collected from Shaanxi Province (China) were first suspended in a series of 10-fold dilutions of sterile water from 10⁻¹ to 10⁻⁶ and plated on agar plates containing the Rhamnolipid Biosurfactant-Specific Screening Medium (RBSSM, per liter): 1 g beef extract, 20 g glucose, 5 g peptone, 0.2 g yeast extract, 0.2 g N,N,N,-trimethyl-1-hexadecane ammonium bromide, 0.005 g methyl blue and 18 g agar. The inoculated plates were incubated at 30° C. for 48 hours. Colonies displaying the anionic biosurfactant-indicating blue coloration with halo around them (Siegmund and Wagner, J. Biotechnol. Tech. 5:265-268, 1991) were selected for further colony purification and confirmation on RBSSM agar plates. Isolated colonies were inoculated into the Biosurfactant Production Liquid Medium (BPLM, pH 7.4) to further confirm and evaluate their surface activities. BPLM was made from the stock solutions and selective carbon sources. BPLM (per liter) contained 5.0 ml phosphate buffer (per liter: 25.82 g K₂HPO₄.3H₂O, 8.7 g KH₂PO₄, 33.4 g Na₂HPO₄.12H₂O, 5.0 g NH₄Cl), 3.0 ml magnesium sulfate solution (22.5 g/L MgSO₄), 1.0 ml calcium chloride solution (36.4 g/L CaCl₂), 1.0 ml ferric chloride solution (0.25 g/L FeCl₃), 1.0 ml trace mineral elements containing MnSO₄ (39.9 mg/L), ZnS O₄.H₂O (42.8 mg/L) and (NH₄)₆Mo₇O₂₄.4H₂O (34.7 mg/L), and one of the following carbon sources: 20 g glucose, 3 g beef extract, 0.2% diesel oil, 0.2% hexane or 0.2% octane (v/v). To measure the surface activity, the liquid cultures were placed in 250 ml Erlenmeyer flasks and incubated at 30° C. on a rotary shaker at 200 rpm. Culture samples (5 ml) were taken over time at 24, 48, 72 and 96 hours. The pure culture, which produced the highest surface activity, was designated as strain NY3 and selected for full characterization.

Genomic DNA preparation, PCR amplification, DNA sequencing and analysis: For mini-preparation of genomic DNA, strain NY3 was grown in 10 ml Tryptone Soya Broth (TSB) medium at 30° C. for 16 hours. Cells were harvested by centrifugation at 4° C. and 4000 rpm for 15 minutes (Beckman JS-21). The supernatant was discarded and the pellet was successively washed once with 10.3% sucrose and twice with 10 mM Tris-HCl and 1 mM disodium ethylenediaminetetraacetate (EDTA), pH 8.0 (TE buffer). The wet cells, equivalent to the volume of 80 μl water, were distributed into 1.5 ml sterile micro-centrifuge tubes. After adding 300 μl of the lysis solution containing 200 μl of 10 mM Tris-HCl and 1 mM EDTA, pH 8.0 and 0.3 M Sucrose (TES buffer), 50 μl of 0.5 M EDTA, 50 μl of lysozyme (50 mg/ml), the tubes were incubated at 37° C. for 30 to 60 minutes until the solution became viscous. Next, 5 μl of proteinase K (20 mg/ml) and 180 μl of 10% sodium dodecyl sulfate (SDS) were added into each tube. After gentle but thorough mixing, the solutions were incubated at 37° C. for 90 minutes. Then, 80 μl of 10% Cetyl Trimethyl Ammonium Bromide (CTAB) was added. After thorough mixing, the tubes were incubated at 65° C. for 10 minutes. The solutions were extracted twice with 600 μl of phenol/chloroform/isoamyl alcohol (25:24:1). The genomic DNA in the upper aqueous phases was recovered and precipitated with 0.6 volumes of isopropanol. The harvested genomic DNA was washed twice with 70% ethanol. After drying at room temperature for 10 minutes, the genomic DNA was dissolved in 50 to 100 μl of sterile water for use in PCR.

The PCR reaction was conducted under conditions described previously (Yin et al., Gene 312:215-224, 2003), except for substitutions in the forward and reverse primers with fD1 and rD1 (Weisburg et al., J. Bacteriol. 173:697-703, 1991) as well as the addition of 1 μg of genomic DNA. Primers used for PCR and DNA sequencing were synthesized by Fisher. The PCR product from the agarose gel was purified using QIAquick® Gel Extraction kits from Qiagen (Valencia, Calif.). DNA sequencing was performed at Oregon State University Center for Genome Research and Biocomputing (CGRB) using the AmpliTaq® dye-terminator sequencing system (Perkin Elmer) and Applied Biosystems automated DNA sequencers (models 373 and 377). Nucleotide sequences were determined for both strands. Sequence analysis was carried out using the Vector NTI® (Invitrogen, Carlsbad, Calif.) software. Nucleotide sequence similarity comparisons were carried out in public databases using the BLAST program (Altschul et al., J. Mol. Biol. 548:403-410, 1990). The 16S rRNA gene sequence of strain NY3 was deposited in GenBank under the accession number GU377209 (incorporated herein by reference, as present in GenBank on Jun. 10, 2011).

Culture conditions for growth of Pseudomonas aeruginosa strain NY3 and production of NY3BS: Strain NY3 was permanently stocked in 20% glycerol solution at −70° C. and temporarily plated and maintained on a Luria-Bertani (LB) agar plate for fresh inoculation of liquid culture. Growth of strain NY3 was evaluated in a series of liquid and solid media. They included the liquid media LB, 2× YT, TSB, YM, YGP and BHI, and the solid media LB, YM, ISP2, ISP4, AS1 and R2YE. Production of NY3 was affected by a number of factors including the concentration of the cells initially inoculated, the media, initial pH, metal ions, cultural temperature, shaking speed and harvest time. For routine production of NY3 biosurfactants (NY3BS), P. aeruginosa strain NY3 was grown in BPLM broth, supplemented with either glucose (BPLMglu) or glycerol (BPLMgly) as the carbon source, at 30° C. on a rotary shaker at 200 rpm for 76 hours.

Characterization of P. aeruginosa strain NY3: For strain characterization, all liquid cultures were inoculated in triplicate in 500 ml Erlenmeyer flasks containing 200 ml BPLM broth or its derivatives at 30° C. on a rotary shaker at 200 rpm. The initial pH values were varied from 2.0 to 10.0 in culture broth in order to determine the optimal pH range for NY3BS production. To determine the optimum carbon source for NY3BS production, glucose (20 g/L), beef extract (5 g/L), hexane (2 ml/L), octane (2 ml/L) and diesel oil (2 ml/L) were alternatively added into BPLM broth as the sole carbon source. Samples were taken at 0, 12, 24, 36, 48, 60, 72, 76, 84, 96 and 102 hours for acquiring surface activity and other measurements.

Measurements of the surface activity of NY3BS: Three methods including oil displacement test, surface tension/critical micelle concentration (CMC) and emulsification activity were employed to evaluate the surface-active properties of NY3BS using either cell free broth (supernatant) or purified NY3BS compounds.

Oil displacement test was conducted as described by Rodrigues et al. (Colloids Surf. B Biointerfaces 49:79-86, 2006). Briefly, a clear round glass plate (20×150 mm) was loaded with 10 ml distilled water and 0.5 ml olive oil in the center and was followed by adding 100 μl supernatant in the center. The centrally located oil was then forced to displace towards the off-center directions while forming a clear oil zone. The concentration of biosurfactant added was proportional to the diameter of the clear zone.

Surface tension was measured by using the maximum bubble pressure method (Kjellin et al., J. Colloid Interface Sci. 262:506-515, 2003). Based on the surface tension measurement, CMC was then obtained by the plot of surface tension and the serial concentration of NY3BS solutions.

Emulsification activity was assessed by following Cooper and Goldenberg (Appl. Environ. Microbiol. 53:224-229, 1987). In brief, a 15-ml graduated clear glass tube with screw cap was filled with 5 ml dimethylbenzene and 5 ml supernatant. After thorough mixing by vortexing at maximum speed for 2 minutes, the tube was left standing undisturbed at room temperature for 24 hours. The height of the dimethylbenzene layer was measured and divided by the total height of dimethylbenzene and aqueous phases. The resulting ratio was multiplied by 100 to obtain the emulsification index E₇₆, which was proportional to the emulsification activity.

For the above measurements, cell free broth was freshly prepared from the NY3BS productive cultures at the time points of 24, 48, 68, 72, 76, 92 and 96 hours, and the purified NY3BS was dissolved in deionized water at the concentration of 1 to 100 mg/L. All measurements were taken in triplicate to minimize the experimental errors and to generate averaged values.

Isolation and purification of NY3BS: 500 ml of the production culture was harvested at 48 hours by centrifugation at 4,000 rpm (Beckman J2-MC) at 4° C. for 15 minutes to remove the cells. The supernatant was acidified to pH 2.0 with concentrated HCl and kept at 4° C. overnight. The precipitate was recovered by centrifugation at 4° C. and 12,000 rpm (Beckman J2-MC) for 30 minutes and then washed twice with aqueous HCl (pH 2.0). The precipitates were dissolved in 1 N NaOH and adjusted to pH 7.0. The solution was dried in lyophilizer. The crude preparation of NY3BS was further extracted twice with CH₂Cl₂ and dried with a rotary evaporator. After the powder was dissolved in 5 ml of 0.01 N NaOH, the solution was filtered with Whatman® No. 4 paper. The filtrate was collected and adjusted to pH 2.0 and then centrifuged at 4° C. and 12,000 rpm for 30 minutes. The pellet was dried with a rotary evaporator to obtain the pure biosurfactant NY3BS which was stored at −20° C. for further analysis.

Structural characterization of NY3BS: MALDI-TOF MS and tandem MS were employed to elucidate the structure of NY3BS. MS analysis was performed by Matrix-Assisted Laser Desorption/Ionization Time-Of-Flight (MALDI-TOF) mass spectrometry using an Applied Biosystems ABI4700 TOF/TOF mass spectrometer in reflector mode with an accelerating voltage of 20 kV. Samples were mixed in a 1:4 ratio with alpha-cyano-4-hydroxycinnamic acid (HCCA) in 50% acetonitrile and 0.1% TFA. An aliquot of 0.5 μl of the sample solution was applied to the sample plate and air dried.

Quantification of the total sugar, protein and rhamnose: Total sugar was determined by the phenol sulfuric acid method according to Dubois et al. (Anal. Chem. 28:350-356, 1956). The standard curve was prepared with D-glucose. Total protein content was measured by Bradford method (Bradford, Anal. Biochem. 72:248-254, 1976), standardized with bovine serum albumin. Rhamnolipid was assessed by quantification of L-rhamnose by the 6-deoxy-hexose method according to Chandrasekaran and Bemiller (Meth. Carbohydr. Chem. 8:89-96, 1980). L-rhamnose was used for making the standard curve.

Effects of temperature and concentrations of salt on the surface activity: To evaluate the effect of temperature variation on NY3BS surface activity, the NY3BS solution at the CMC was heated to 40° C., 60° C., 80° C. and 100° C. in water bath, or 120° C. and 140° C. by autoclave for 1 hour. After cooling to room temperature, the corresponding surface tensions were measured to evaluate the NY3BS thermal stability. The NY3BS solution at the CMC was added with serial concentrations of 4%, 8%, 12%, 16%, 20%, and 24% NaCl solutions. After thorough mixing, the corresponding surface tensions were measured to evaluate NY3BS tolerance to salt.

Assay for PAH degradation by P. aeruginosa strain NY3: The seed culture of strain NY3 was prepared by inoculating a single colony into a 125 ml Erlenmeyer flask containing 30 ml BPLMGlu broth as the sole carbon source. The culture was incubated at 30° C. on a rotary shaker at 200 rpm. When the optical density at 600 nm reached 0.5, a 10 ml culture was transferred to a 500 ml Erlenmeyer flask containing 100 ml BPLM broth. The broth was supplemented with a mixture of equal amounts of the following polycyclic aromatic hydrocarbons: fluorene, anthracene, phenanthrene, pyrene and fluoranthene to the final concentration of 25 mg/L (5 mg/L for each). Triplicate cultures, including one negative control with autoclaved cells of strain NY3 added, were incubated at 30° C. on a rotary shaker at 200 rpm. The culture samples were taken at the time points of 0, 1.5, 12, 15, 18, 21 and 24 hours for analysis. The residual PAHs in the cultures were recovered by three repeated extractions with cyclohexane and followed by dehydration using anhydrous Na₂SO₄. After passage through a 0.45 μm membrane filter, the preparations were concentrated on a rotary evaporator. The pellets were dissolved in methanol and quantified by HPLC (JASCO LC-2000 chromatograph equipped with a diode-array UV-visible detector). The samples were analyzed at 25° C. by injecting 20 μl into a reverse-phase ODS-C₁₈ column (5 μM, 250×4.6 mm) and using isocratic elution with the mobile phase of 15% H₂O and 85% methanol at a flow rate of 1 ml/min. Elution of PAHs was monitored at 254 nm. The residual concentrations for each PAH compound were quantified by comparison of the peak areas between the sample and the control

### Example 2

### Isolation and Characterization of Pseudomonas aeruginosa Strain NY3

Serial dilutions of the petroleum-contaminated soil samples in sterile water were screened on the solid medium RBSSM. After two days of incubation at 30° C., seven large, flat, smooth, colonies of rod-shaped bacteria produced visual rhamnolipids as indicated by the presence of blue halos (Siegmund and Wagner, J. Biotechnol. Tech. 5:265-268, 1991). These colonies were further purified on RBSSM agar plates according to their uniform growth, color, morphological and microscopic characteristics. To confirm their abilities to produce the biosurfactants in liquid culture, individual colonies were inoculated in BPLMGlu medium. Production of biosurfactants in these cultures was monitored by measuring the surface tension and emulsification activity. Among them, one pure culture, designated as strain NY3, producing the lowest surface tension (32.8 mN/m²) and highest emulsification activity (E₇₆=100%), was selected for in-depth characterization.

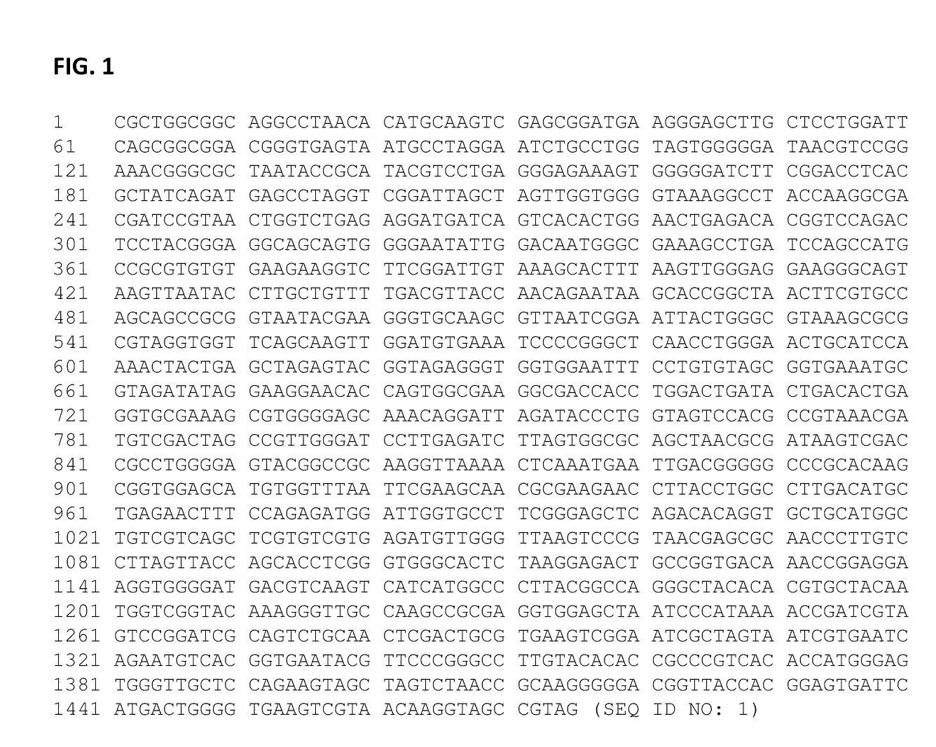

Sequencing of the 16S rRNA gene for an unknown pure microorganism has appeared as the predominant strategy in the literature for strain classification. By adopting the published primers fD1 and rD1 for most eubacteria (Weisburg et al., J. Bacteriol. 173:697-703, 1991), and using genomic DNA of strain NY3 as a PCR template, the 1.5 kb fragment was successfully amplified. The gel-purified PCR product was directly submitted for sequencing using the PCR primers mentioned above. The 1475 by sequence (FIG. 1; SEQ ID NO: 1) was obtained and analyzed by BLAST search against GenBank database (Altschul et al., J. Mol. Biol. 548:403-410, 1990). It revealed the high similarity to the 16S rRNA genes from Pseudomonas aeruginosa strains (e.g., GenBank Accession Nos.: EF062513 (100% identity), GQ180118, GQ180117, FJ948174 and FM209186 (99% identities)). Based on the BLAST result, morphological and microscopic characteristics, the pure isolate was classified as P. aeruginosa strain NY3.

The growth of strain NY3 was evaluated on agar plates made from different media. Those include solid media LB for E. coli, ISP2, ISP4, R2YE, AS1 and YM for Streptomyces. After two days incubation at 30° C., robust growth was observed on ISP2, YM, AS1 and LB. However, no growth was observed on ISP4 and R2YE. A coffee-brownish color was visualized when grown on YM. The growth of strain NY3 was also tested in the different liquid media. Those include LB and 2×YT for E. coli, YM and TSB for Streptomyces, YGP for yeast and BHI for Paenibacillus. Strain NY3 was able to grow well in all these liquid media.

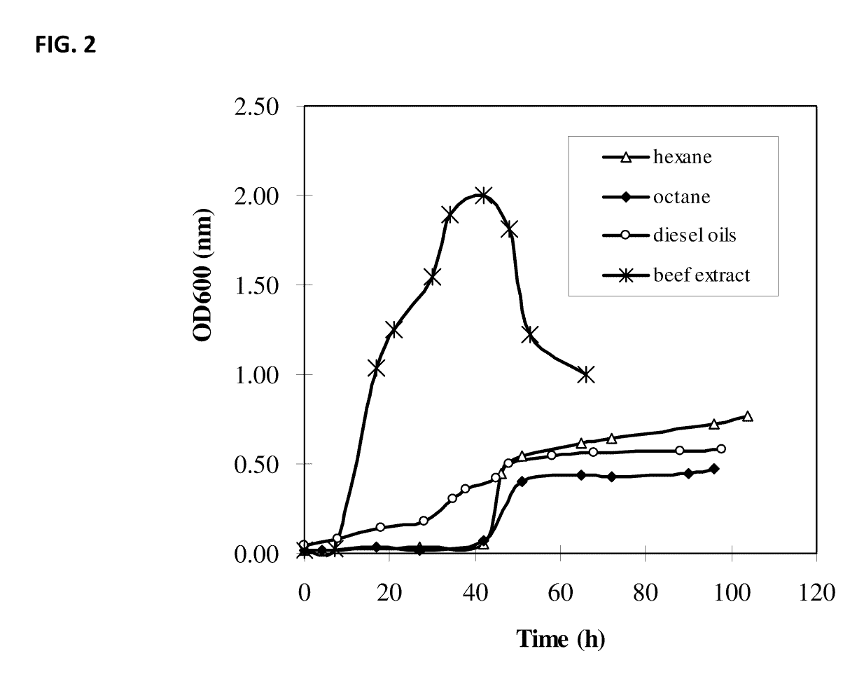

To optimize the fermentation conditions for growth and biosurfactant production, strain NY3 was cultivated in BSPM supplemented with different carbon sources and at various initial pH. FIG. 2 demonstrates that strain NY3 is capable of utilizing n-alkanes as sole carbon and energy sources. The growth rate with hexane was superior to diesel oil and octane. The maximum growth of strain NY3 with hexane was approximately three times lower than that with beef extract. Regardless of whether glucose (20 g/L), glycerol (20 g/L), or beef extract (3 g/L) was used as the sole carbon source, the growth curves were very similar. However, the culture supernatant from the glucose fermentation produced higher surface activity than those grown with either beef extract or glycerol. Varying the initial pH from 2 to 10 in the BSPMGlu, the best initial pH for NY3BS production and surface activity was determined as 9.0. Under the optimum fermentation conditions, the lowest surface tension (32.8 mN/m2), the best emulsification activity (E₇₆=100%) and the maximum oil displacement ability (10 cm) for strain NY3 were simultaneously achieved when the measurement was conducted with the cell free culture or with purified biosurfactant NY3BS prepared from 76 hour culture samples (Table 2). In addition, the yield of NY3BS produced by strain NY3 was determined to be 0.2 g/L after 76 hour fermentation in BSPMGlu medium.

TABLE 2
Surface activity of rhamnolipid biosurfactant NY3BS

| | Time (h) | | | | | | |
|---|---|---|---|---|---|---|---|
| | 24 | 48 | 68 | 72 | 76 | 92 | 96 |
| E₇₆ (%) | 70 | 100 | 100 | 82 | 100 | 100 | 90 |
| R (cm) | 1.0 | 2.0 | 5.0 | 6.0 | 8.0 | 8.0 | 4.0 |
| Surface | 52.31 | 42.46 | 34.63 | 41.92 | 32.81 | 34.98 | 41.92 |
| tension | | | | | | | |
| (mN/m²) | | | | | | | |

### Example 3

### Degradation of PAHs by Strain NY3

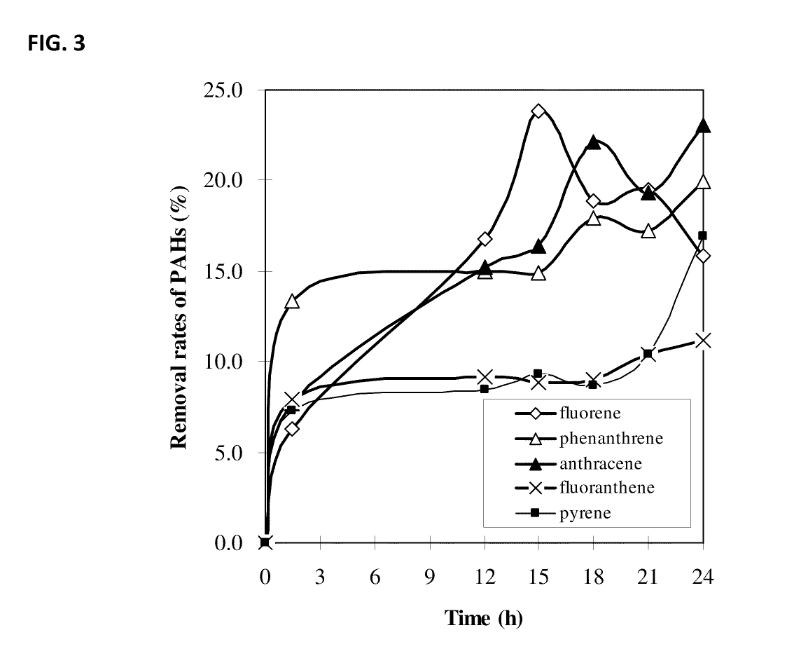

A mixture of five compounds (fluorene, anthracene, phenanthrene, pyrene and fluoranthene) were employed to evaluate the capacity of the in vivo degradation of the polycyclic aromatic hydrocarbons by strain NY3. They were added into the liquid medium BSPM, which had been previously inoculated with strain NY3 or dead NY3 cells as negative control. The residual compounds were recovered from the fermentation samples collected at different time points by extraction with organic solvent. Quantitative analysis of the residual PAHs by HPLC are shown in FIG. 3. By the end of 24 hours, 23.1% anthracene, 19.9% phenanthrene, 16.9% pyrene, 15.8% fluorine, and 11.2% fluoranthene were removed. In general, strain NY3 was capable of degrading all five PAH substrates although their removal rates were different (FIG. 3). The degradation rates for three-ring PAHs, including fluorene, phenanthrene and anthracene, were higher than four-ring PAHs like fluoranthene and pyrene. There were no significant differences in the degradation rates among the three-ring PAHs. The removal rates for three-ring PAHs gradually increased over fermentation time while the removal rates for four-ring PAHs showed no obvious changes between 1.5 hours and 18 hours. In addition, the removal rate for each PAH was relatively high during the first 1.5 hours of fermentation. Without being bound by theory, it is believed that during the time period 0.1 hours through 1.5 hours, a portion of the removal rate may have been contributed by the NY3 cells trapping the PAH. Thus, only a portion of the removal rate observed may be due to degradation by the NY3 cells.

### Example 4

### Characterization of Biosurfactant NY3BS

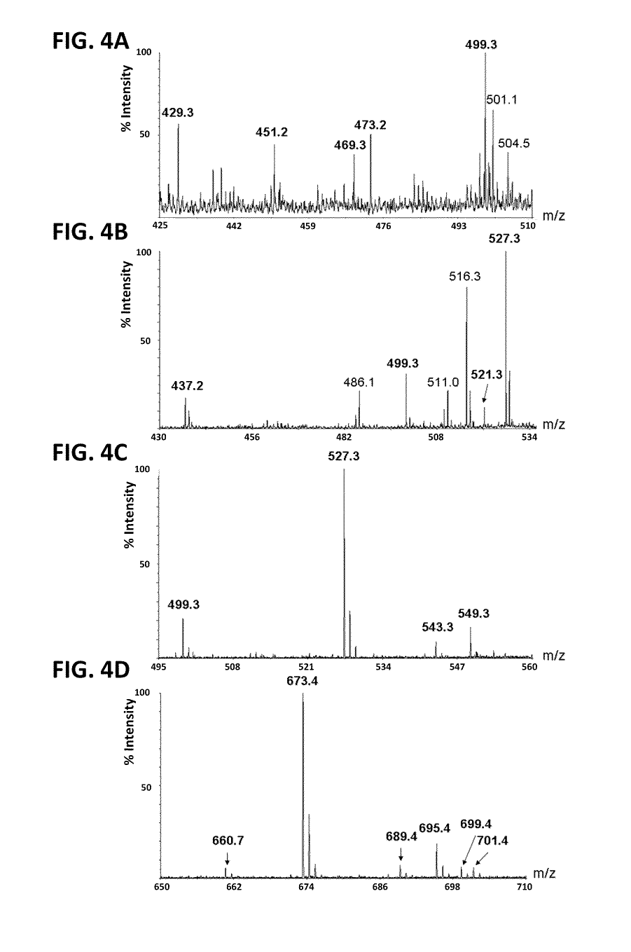

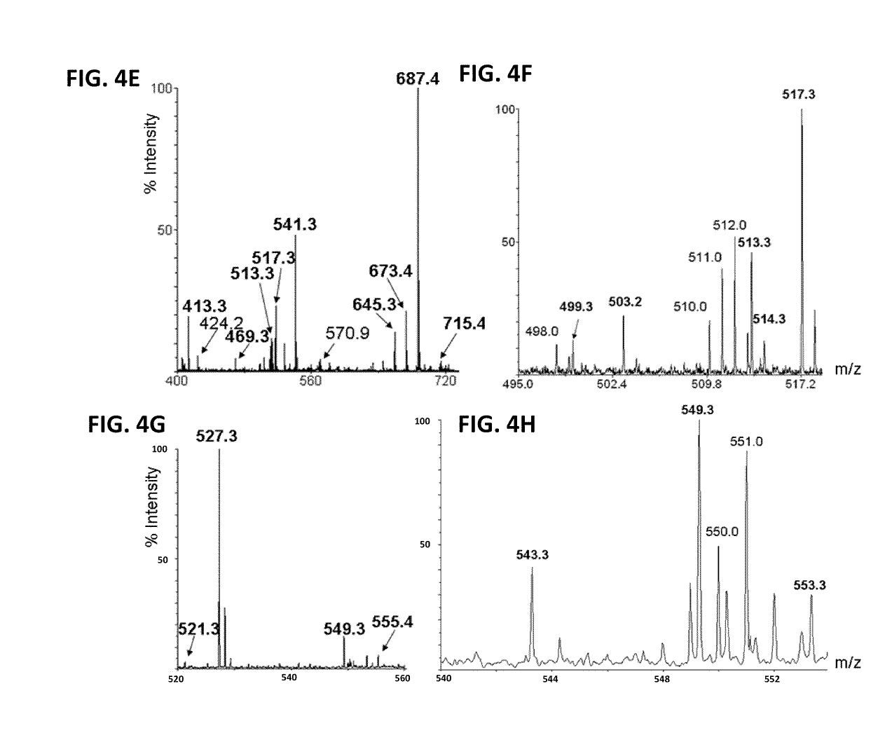

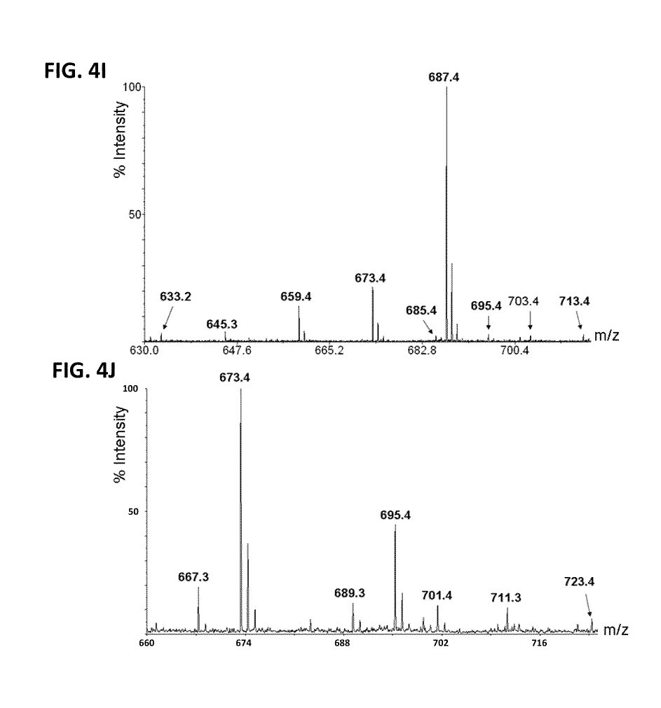

NY3BS was extracted from a 76 hour fermentation in BSPMGlu or BSPMbspmGly media. The purified NY3BS was analyzed for sugar and protein contents by the phenol sulfuric acid and Bradford methods, respectively. The results indicated NY3BS contained 63.4% total sugar, 34.6% rhamnose, and 0.35% protein. Purified NY3BS was analyzed by MALDI-TOF MS and tandem mass spectrometry. The results are summarized in Table 3 and shown in FIG. 4. A total of 25 components of rhamnolipid biosurfactant NY3BS, which represented 37 different metal ion (Na⁺ and/or 2Na⁺ or K⁺) adducts, were detected by MALDI-TOF MS. The parent ions at m/z 527.3 and 673.4 were dominant and could be assigned to singly sodiated monorhamnolipid [Rha-C₁₀-C₁₀+Na]⁺ and dirhamnolipid [Rha-Rha-C₁₀-C₁₀+Na]⁺, respectively (FIG. 4A to J). The parent ions at m/z 499.3 (FIG. 4A), 687.4 (FIGS. 4E and I), 513.3 and 517.3 (FIG. 4F), 549.3 (FIG. 4H) and 695.4 (FIG. 4J), were less abundant and could be assigned to [Rha-C₁₀-C₈+Na]⁺, [Rha-C₁₀-C_{10:1}+K]⁺, [Rha-Rha-C₁₀-C_{10:1}+K]⁺, [Rha-C₁₀-C_{8:1}+K]⁺, [Rha-Rha-C₁₀+K]⁺, [Rha-C_{24:1}+Na]⁺ and [Rha-Rha-C₁₀-C₁₀−H+2 Na]⁺, respectively (Table 3).

TABLE 3
Molecular ions observed in rhamnolipid biosurfactant NY3BS

| | Calcd | | | | | | |
|---|---|---|---|---|---|---|---|
| Molecular | Mass | [M + Na]⁺ | | [M + K]⁺ | | [M − H + 2Na]⁺ | |
| formula | units [M] | Obsd | Calcd^{a} | Obsd | Calcd | Obsd | Calcd |

Monorhamnolipids

| | | | | | | | | |
|---|---|---|---|---|---|---|---|---|
| Rha-C₈-C_{8:1} | C₂₂H₃₈O₉ | 446.25158 | 469.3 | 469.24135 | — | 485.21529 | — | 491.2233 |
| Rha-C₁₀-C₈ | C₂₄H₄₄O₉ | 476.29853 | 499.3 | 499.2883 | — | 515.26224 | 521.3 | 521.27025 |
| Rha-C₁₀-C_{8:1} | C₂₄H₄₂O₉ | 474.28288 | — | 497.27265 | 513.3 | 513.24659 | — | 519.26483 |
| Rha-C₁₀-C₁₀ | C₂₆H₄₈O₉ | 504.32983 | 527.3 | 527.3196 | 543.3 | 543.29354 | 549.3^{b} | 549.30155 |
| Rha-C₁₀-C_{10:1} | C₂₆H₄₆O₉ | 502.31418 | — | 525.30395 | 541.3 | 541.27789 | — | 547.2859 |
| Rha-C₁₀-C₁₂ | C₂₈H₅₂O₉ | 532.36113 | 555.4 | 555.3509 | — | 571.32484 | — | 554.33285 |
| Rha-C₁₀-C_{12:1} | C₂₈H₅₀O₉ | 530.34548 | 553.3 | 555.33525 | — | 569.30919 | — | 575.3172 |
| Rha-C_{8:1} | C₂₀H₃₄O₁₁ | 450.21011 | 473.2 | 473.19988 | — | 489.17382 | — | 495.18183 |
| Rha-C₁₆ | C₂₂H₄₀O₇ | 416.2774 | 439.1 | 439.26717 | — | 455.24111 | — | 461.24912 |
| Rha-C_{16:1} | C₂₂H₃₈O₇ | 414.26175 | 437.2 | 437.25152 | — | 453.22546 | — | 459.23347 |
| Rha-C_{17:1} | C₂₃H₄₀O₇ | 428.2774 | 451.2 | 451.26717 | — | 467.24111 | 473.2 | 473.24912 |
| Rha-C_{24:1} | C₃₀H₅₄O₇ | 526.38695 | 549.3^{b} | 549.37672 | | 565.35066 | — | 571.35867 |

Dirhamnolipids

| | | | | | | | | |
|---|---|---|---|---|---|---|---|---|
| Rha-Rha-C₆-C_{6:1} | C₁₈H₃₀O₉ | 390.18898 | 413.3 | 413.17875 | 429.3 | 429.15269 | — | 435.17093 |
| Rha-Rha-C₈-C₈ | C₂₈H₅₀O₁₃ | 594.32514 | | 617.31491 | 633.2 | 633.28885 | — | 639.29686 |
| Rha-Rha-C₁₀-C₈ | C₃₀H₅₄O₁₃ | 622.35644 | 645.3 | 645.34621 | 660.7 | 661.32015 | 667.3 | 667.32816 |
| Rha-Rha-C₁₀-C_{8:1} | C₃₀H₅₂O₁₃ | 620.34079 | — | 643.33056 | 659.4 | 659.3045 | — | 665.31251 |
| Rha-Rha-C₁₀-C₁₀ | C₃₂H₅₈O₁₃ | 650.38774 | 673.3 | 673.37751 | 689.6 | 689.35145 | 695.4^{c} | 695.35946 |
| Rha-Rha-C₁₀-C_{10:1} | C₃₂H₅₆O₁₃ | 648.37209 | — | 671.36186 | 687.4 | 687.3358 | — | 693.34381 |
| Rha-Rha-C_{10:1}-C_{10:1} | C₃₂H₅₄O₁₃ | 646.35644 | — | 669.34621 | 685.4 | 685.32015 | — | 691.32816 |
| Rha-Rha-C₁₀-C₁₂ | C₃₄H₆₂O₁₃ | 678.41904 | 701.4 | 701.40881 | — | 717.38275 | 723.4 | 723.39076 |
| Rha-Rha-C₁₀-C_{12:1} | C₃₄H₆₀O₁₃ | 676.40339 | 699.4 | 699.39316 | 715.4 | 715.3671 | — | 721.37511 |
| Rha-Rha-C_{9:1} | C₂₁H₃₆O₁₁ | 464.22576 | — | 487.21553 | 503.2 | 503.18947 | — | 509.19748 |
| Rha-Rha-C₁₀ | C₂₂H₃₈O₁₁ | 478.24141 | — | 501.23118 | 517.3 | 517.20512 | — | 523.21313 |
| Rha-Rha-C₂₄ | C₃₆H₆₈O₁₁ | 674.46051 | — | 697.45028 | 713.4 | 713.42422 | — | 719.43223 |
| Rha-Rha-C_{24:1} | C₃₆H₆₆O₁₁ | 672.44486 | 695.4^{c} | 695.43463 | 711.3 | 711.40857 | — | 717.41658 |

| Parent molecular | |
|---|---|
| ions | Daughter ions |
| [Rha-C₁₀-C₁₀ + | 80.0, 83.0, 95.0, 96.0, 111.0, 113.0, 169.0, 185.0, 193.1, 197.9, 209.1, 211.1, 281.1, |
| Na]⁺ at m/z 527.3 | 295.2, 308.3, 321.2, 335.2, 351.1, 357.2, 368.9, 381.2, 409.2 |
| [Rha₂-C₁₀-C₁₀ + | 71.0, 80.0, 85.0, 95.0, 111.0, 113.0, 153.0, 169.0, 185.0, 193.1, 211.1, 265.1, 279.2, |
| Na]⁺ | 281.1, 295.2, 308.3, 315.1, 321.2, 331.1, 333.1, 359.2, 381.3, 409.3, 495.1, 503.2, |
| at m/z 673.3 | 517.0, 527.3, 555.4 |

^{a}Calculated monoisotopic masses.
^{b,c}The identical mass units were detected for different compounds. They could be distinguished from each other only by analysis with the higher resolution mass spectrometry facilities.

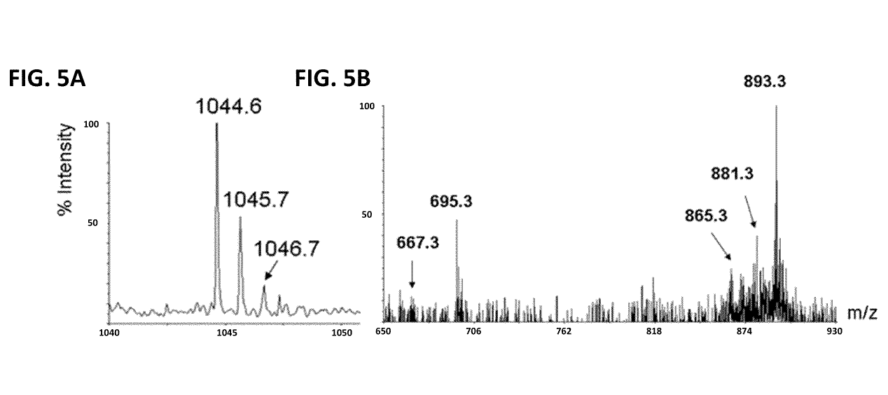

Many minor or trace components of rhamnolipid NY3BS were also observed (Table 3 and FIG. 4A to J). Among them were ten novel rhamnolipids, which included five monorhamnolipids: Rha-C₈-C_{8:1}, Rha-C₁₆, Rha-C_{16:1}, Rha-C_{17:1} and Rha-C_{24:1}, and five dirhamnolipids: Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}. In addition, MALDI-TOF MS revealed an unusually large molecular ion at m/z 1044.6 (FIG. 5A). The corresponding NY3BS sample was isolated from the fermentation using glycerol as the sole carbon source. Further tandem MS analysis of this parent ion gave fragment ions in which one strong signal at m/z 695.4 corresponded to a known rhamnolipid component: doubly sodiated dirhamnolipid [Rha-Rha-C₁₀-C₁₀−H+2Na]⁺ while one weak signal at m/z 667.3 matched another known rhamnolipid component: doubly sodiated dirhamnolipid [Rha-Rha-C₁₀-C₈−H+2 Na] (FIG. 5B). Moreover, tandem MS data for the parent ions at m/z 527.3 and 673.3 were obtained and are summarized in Table 3. The fragment ions from the parent ions at m/z 673.4 gave recognizable ions with the same mass units as the parent ions at m/z 527.3 (FIG. 4C) and 555.4 (FIG. 4G).

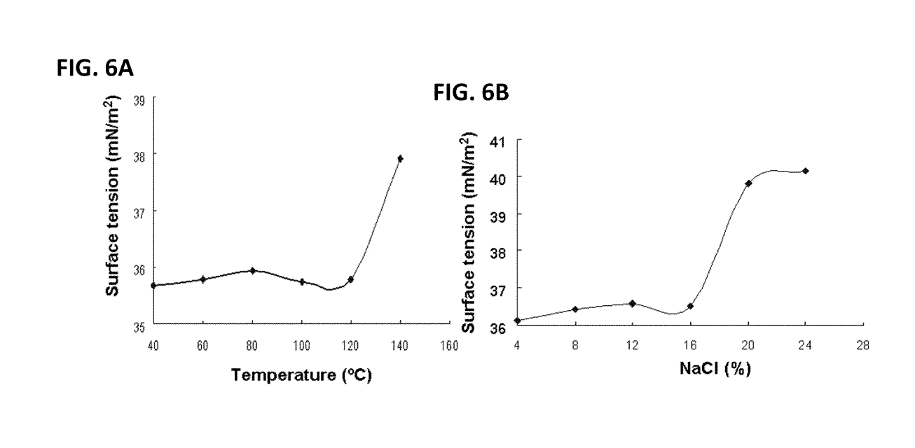

The surface tension was measured with NY3BS solutions treated at various temperatures. Rhamnolipid NY3BS was resistant to a wide range of temperatures. No significant changes were observed in the surface tension after 1 hour at 120° C. The surface tension increased from 32.8 to 38.0 mN/m² after 1 hour at 140° C. (FIG. 6A). NY3BS was still effective in the presence of a high concentration of sodium chloride. The surface tension remained less than 35 mN/m² even though the concentration of NaCl was elevated to 16%. However, the surface tension rapidly increased to a high of 43 mN/m² when the final concentration of NaCl reached 20% (FIG. 6B).

### Example 5

### Anti-Microbial Activity of NY3BS

NY3BS was prepared as described in Example 1 in sterile deionized water at a concentration of 28 g/L and stored at −20° C. until use.

Three-day pre-cultures (20 ml) of cyanobacteria Synechocystis PCC6803 or Synechocystis UTEX 2470 were used to inoculate 13 ml of BG11 medium. The cultures were incubated in a light-controlled incubator oven (Hoffman Manufacturing, Albany, Oreg.) at 28° C. for 3 days. Cultures were treated with 0, 2.8, 5.6, or 14 mg of NY3BS preparation. NY3BS reduced Synechocystis growth at 2.8 mg and almost completely inhibited cell growth at the 14 mg dose.

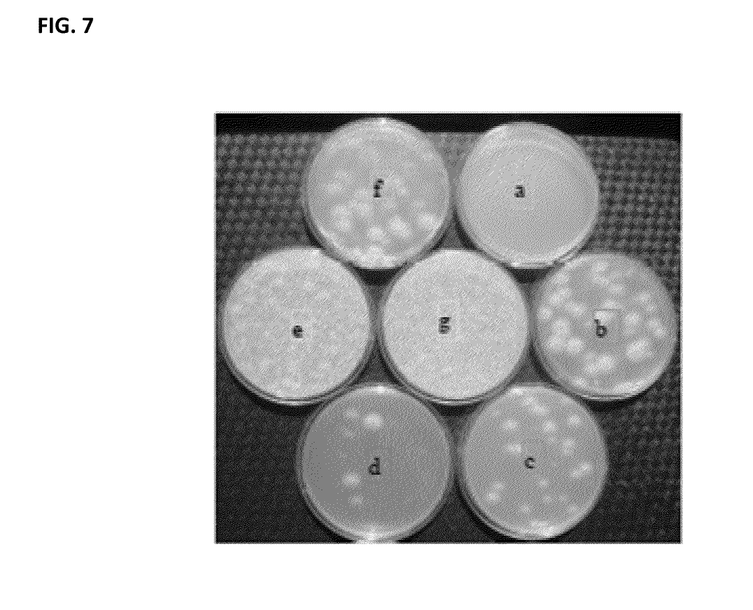

Potato dextrose agar (PDA) plates were prepared with 0, 1.4, 2.8, or 4.2 mg of NY3BS preparation. The plates were inoculated with Fusarium oxysporum and growth was observed after 3 days at 30° C. All doses of NY3BS substantially reduced Fusarium growth (FIG. 7). Growth of Fusarium oxysporum was completely inhibited on PDA plates with 90 mg/L of NY3BS.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A composition comprising one or more rhamnolipids selected from Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}.

2. The composition of claim 1, wherein the one or more rhamnolipids comprise each of Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}.

3. The composition of claim 2, further comprising one or more rhamnolipids listed in Table 3.

4. The composition of claim 1, wherein the one or more rhamnolipids are isolated from Pseudomonas aeruginosa.

5. The composition of claim 4, wherein the one or more rhamnolipids are isolated from P. aeruginosa strain NY3.

6. The composition of claim 1, further comprising a carrier, an antimicrobial agent, a non-rhamnolipid surfactant, or a combination of two or more thereof.

7. A method for producing one or more rhamnolipids of Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha-C_{10:1}-C_{10:1}, Rha-Rha-C₂₄, and Rha-Rha-C_{24:1}, comprising cultivating Pseudomonas aeruginosa under conditions sufficient to produce the one or more rhamnolipids.

8. The method of claim 7, wherein the conditions sufficient to produce the one or more rhamnolipids comprise cultivating the P. aeruginosa in a liquid medium comprising glucose, glycerol, beef extract, hexane, octane, diesel oil, or a combination of two or more thereof as the carbon source.

9. The method of claim 8, wherein the initial pH of the liquid medium is about 9.0.

10. The method of claim 7, wherein the P. aeruginosa comprises P. aeruginosa strain NY3.

11. The method of claim 7, further comprising isolating the one or more rhamnolipids from the culture.

12. A method of treating an environmental material contaminated with one or more of a hydrocarbon, heavy metal, or pesticide, comprising contacting the environmental material with an effective amount of the composition of claim 1.

13. The method of claim 12, wherein the environmental material comprises soil, sediment, sludge, water, or a combination thereof.

14. The method of claim 12, wherein the hydrocarbon comprises a polycyclic aromatic hydrocarbon.

15. The method of claim 14, wherein the polycyclic aromatic hydrocarbon comprises fluorene, anthracene, phenanthrene, pyrene, or fluoranthene.

16. A method of inhibiting microbial growth, comprising contacting the microbe with an effective amount of the composition of claim 1.

17. The method of claim 16, wherein the microbe comprises one or more bacteria, cyanobacteria, or fungi.

18. The method of claim 17, wherein the microbe is Fusarium oxysporum or Synechocystis.

19. The method of claim 16, wherein contacting the microbe with the composition comprises administering the composition to a mammal or a plant.

20. A method of treating an environmental material contaminated with hydrocarbons, comprising contacting the environmental material with a preparation of biosurfactants comprising one or more of Rha-C₈-C_{8:1}, Rha-C₁₆, Rha-C_{16:1}, Rha-C_{17:1}, Rha-C_{24:1}, Rha-Rha-C₆-C_{6:1}, Rha-Rha-C_{9:1}, Rha-Rha C_{10:1}-C_{10:1}, Rha-Rha-C₂₄, and Rha-Rha-C_{24:1}, thereby treating the environmental material.

21. The method of claim 20, wherein the preparation of biosurfactants is produced by:
collecting a supernatant from a P. aeruginosa strain NY3 culture;
acidifying the supernatant; and
recovering a precipitate, thereby producing a crude biosurfactant preparation.

22. The method of claim 21, further comprising:
extracting the crude biosurfactant preparation with methylene chloride;
acidifying the extracted biosurfactant preparation; and
collecting the resulting precipitate, thereby producing a purified biosurfactant preparation.

23. A Pseudomonas aeruginosa strain NY3 bacterium, wherein the bacterium produces the rhamnolipids listed in Table 3 following 76 hour fermentation at 30° C. in medium containing (per liter): 5.0 ml phosphate buffer (25.82 g/L K₂HPO₃.3H₂O; 8.7 g/L KH₂PO₄; 33.4 g/L Na₂HPO₄.12H₂O; 5.0 g/L NH₄Cl), 3.0 ml MgSO₄ solution (22.5 g/L MgSO₄), 1.0 ml CaCl₂ solution (36.4 g/L CaCl₂), 1.0 ml FeCl₃ solution (0.25 g/L FeCl₃), 1.0 ml trace mineral elements (39.9 mg/L MnS O₄; 42.8 mg/L ZnS O₄.H₂O; 34.7 mg/L (NH₄)₆MO₇O₂₄.4H₂O), and 20 g/L glucose.
